(19) United States
(12) Patent Application Publication
(10) Pub. No.: US 2023/0008929 A1
(43) Pub. Date: Jan. 12, 2023

(54) Assembly of a Containment Means with an Aseptic Working Chamber

(71) Applicant: Skan AG, Allschwil (CH)

(72) Inventors: Frank Martin Lehmann, Binningen (CH); Yves Scholler, Hegenheim (FR); Matthias Sommerhalder, Bättwil (CH)

(73) Assignee: Skan AG, Allschwil (CH)

(21) Appl. No.: 17/783,400

(22) PCT Filed: Nov. 30, 2020
(86) PCT No.: PCT/CH2020/000018

§ 371 Date: Jun. 8, 2022

(30) Foreign Application Priority Data
Dec. 10, 2019 (EP) 19405020.9

(51) Int. Cl. B01L 1/04; A61L 2/22; B01D 46/12; B01D 46/62; B08B 15/02

(52) U.S. Cl. CPC B01L 1/04; A61L 2/22; B01D 46/12; B01D 46/62; B08B 15/023; A61L 2202/122; B01D 2273/30; B01L 2200/087; B01L 2300/0681; B08B 2215/003

(57) ABSTRACT

The subject matter is the assembly of a containment means with an aseptic working chamber and associated decontamination arrangement. The working chamber is delimited at the bottom by a base, and above the working chamber is a circulating air zone, in which a circulating air filter with a circulating air fan is arranged. The circulating air zone and the working chamber are delimited towards the outside by a housing. At least a first returning air channel leads from the working chamber into the circulating air zone. A returning air filter is fluidically connected to the returning air channel, specifically is arranged facing the circulating air zone. The returning air filter can be arranged at the opening of the returning air channel into the circulating air zone or arranged in the circulating air channel, set back from the opening. The returning air filter and the circulating air filter are in the form of a plate filter or an exchangeable filter cartridge or a replaceable filter insert. A single returning air filter or a series of interacting returning air filters and a single circulating air filter or a series of interacting circulating air filters can be installed. Containment means in which larger volumes of air are to be processed also have a second returning air channel at or in which a returning air filter is also installed.

### FIELD OF THE INVENTION

The present invention relates to the construction of a containment means with an aseptic working chamber, and to an associated decontamination arrangement.

The working chamber is delimited at the bottom by a base, and above the working chamber is a circulating air zone, in which a circulating air filter with a circulating air fan is arranged. The circulating air zone and the working chamber are delimited towards the outside by a housing. At least a first returning air channel leads from the working chamber into the circulating air zone. A returning air filter is fluidically connected to the returning air channel. Such systems are used for the treatment of sterile products, in which no contaminating particles from outside must reach the product to be treated. Containment means are in particular isolators, for example for the pharmaceutical-chemical industry. The term further includes all types of RABS (Restricted Access Barrier System).

### PRIOR ART

EP 3 170 545 A1 in the name of the applicants discloses an example of the construction of a containment means. On the front side, glove ports are inserted in a transparent window, so that the operator can see into the working chamber and access it using the gloves. The laminar air flow introduced into the working chamber is guided via a returning air channel, purified by filter units, into the circulating air zone situated above the working chamber. The filter units, for example in the form of filter cartridges, are inserted into the returning air channel at the widened inlet and thus permit advantageous handling when changing the filter. However, if the widened inlet was any larger, the ergonomics for the operator at the working position in front of the containment means would not be able to be configured wholly optimally.

EP 2 666 532 B1 discloses a slightly different construction of a containment means. Here, two returning air channels are provided for returning the laminar air flow introduced into the working chamber into the circulating air zone arranged above the working chamber, and a filter unit which rests on the base of the working chamber is installed at the inflow opening of each of these returning air channels. However, the space available in the working chamber for equipment positioned therein is thus restricted, and the operator must reach over the filter unit, effectively stretch further forward, with the work gloves in order to reach the intended position, for example a processing machine.

### Object of the Invention

Proceeding from the prior-known prior art, the object underlying the invention is to make the structural construction of a containment means with an aseptic working chamber more ergonomically optimal for the operators working at the containment means and thereby use the space available in the working chamber in the best possible way for the operation and setting up of equipment. Finally, the cleanroom concept is to lead to efficient operating and system costs.

Overview of the invention

The invention relates to the construction of a containment means with an aseptic working chamber and an associated decontamination arrangement. The working chamber is delimited at the bottom by a base, and above the working chamber is a circulating air zone, in which a circulating air filter with a circulating air fan is arranged. The circulating air zone and the working chamber are delimited towards the outside by a housing. At least a first returning air channel leads from the working chamber into the circulating air zone. A returning air filter is fluidically connected to the returning air channel, specifically is arranged facing the circulating air zone.

Specific embodiments of the invention are defined hereinbelow: The returning air filter can be arranged at the opening of the returning air channel into the circulating air zone or in the returning air channel, set back from the opening. The opening of the returning air channel holding the returning air filter can be in the form of a projection which widens the circulating air zone or projects into the circulating air zone, or it merges into the circulating air zone without a projection, virtually with a linear cross-section.

The returning air filter and the circulating air filter are in the form of a plate filter or an exchangeable filter cartridge or a replaceable filter insert. A single returning air filter or a series of interacting returning air filters can be provided, or a single circulating air filter or a series of interacting circulating air filters can be installed.

The at least a first returning air channel comprises as components a transparent front window and a transparent channel window arranged spaced apart therefrom, and has an inflow opening which is situated above and close to the base.

Between the circulating air zone and the working chamber there extends an air guide element which serves to generate a parallel displacement flow flowing downwards in the working chamber (laminar flow).

The decontamination arrangement has a vaporizer positioned in the circulating air zone, or at least one atomizing nozzle. Decontamination agent is supplied to the vaporizer or the atomizing nozzle via a material line by means of a pump, wherein the atomizing nozzle sprays the decontamination agent in the form of an aerosol with compressed air.

5

The at least one atomizing nozzle is directed into:
the working chamber; and/or
an intermediate space which is situated between the circulating air zone and the air guide element; and/or
the at least a first returning air channel.

During operation of the containment means in decontamination mode with the vaporizer as part of the decontamination arrangement, the circulating air fan is controlled with reduced power, e.g. 50%.

15

Depending on the size and purpose of the working chamber, a series of atomizing nozzles directed into the working chamber can be provided. During operation of the containment means in decontamination mode with the atomizing nozzles in the decontamination arrangement, the circulating air fan is down-regulated at least to such an extent that no volume flow is conveyed. Advantageously, the atomizing nozzles are arranged offset with respect to one another such that the aerosol introduced as the decontamination agent circulates in the working chamber.

The construction is further completed by a washing nozzle directed into:
the working chamber; and/or

the intermediate space which is situated between the circulating air zone and the air guide element; and/or
the at least a first returning air channel, specifically upstream of the returning air filter contrary to the direction of flow.

The washing nozzles directed into the returning air channel and/or into the intermediate space are preferably fixedly installed, while the washing nozzle directed into the working chamber can alternatively be seated on a manual spray gun.

In particular, containment means which have a specific purpose or in which larger air volumes are to be processed additionally have a second returning air channel, which comprises a wall portion of the housing and a channel wall spaced apart therefrom, and which has an inflow opening which is again situated above and close to the base. The returning air filter belonging to the second returning air channel is also arranged facing the circulating air zone, specifically at the opening of the second returning air channel into the circulating air zone or directly in the second returning air channel. The opening of the second returning air channel which holds the returning air filter can likewise be in the form of a projection which widens the circulating air zone or projects into the circulating air zone, or it merges into the circulating air zone without a projection, virtually with a linear cross-section. As for the first returning air channel, the returning air filter for the second returning air channel is also in the form of a plate filter or an exchangeable filter cartridge or a replaceable filter insert.

Also where two returning air channels are present, the circulating air filter can be in the form of a plate filter or exchangeable filter cartridge or replaceable filter insert. A single returning air filter or a series of interacting returning air filters, or a single circulating air filter or a series of interacting circulating air filters can thereby be installed. As for the first returning air channel, an atomizing nozzle and a washing nozzle can also be installed for the second returning air channel.

### BRIEF DESCRIPTION OF THE ACCOMPANYING DRAWINGS

The construction according to the invention of a containment means with an aseptic working chamber is shown in different embodiments, in each case in the form of a schematic diagram, namely:

FIG. 1—a first embodiment with a returning air channel having a projection projecting into the circulating air zone, a returning air filter installed therein, a circulating air filter, and a vaporizer as a component of the decontamination arrangement;

FIG. 2—a second embodiment having the construction according to FIG. 1 with two returning air channels;

FIG. 3—a third embodiment with a returning air channel having a projection which widens the circulating air zone, a returning air filter installed therein, a circulating air filter, and atomizing nozzles as a component of the decontamination arrangement;

FIG. 4—a fourth embodiment with two returning air channels, each having a projection projecting into the circulating air zone, a returning air filter installed therein, a circulating air filter, and atomizing nozzles as a component of the decontamination arrangement;

FIG. 5—a fifth embodiment having the construction according to FIG. 4 with two returning air channels, each having a projection which widens the circulating air zone, a returning air filter installed therein, and atomizing nozzles as a component of the decontamination arrangement;

FIG. 6—a sixth embodiment having the construction according to FIG. 5 with in each case an exchangeable filter cartridge as the returning air filter; and

FIG. 7—a seventh embodiment with two returning air channels each with an exchangeable filter cartridge as the returning air filter, exchangeable filter cartridges arranged in series as the circulating air filter, and atomizing nozzles as a component of the decontamination arrangement.

### EXEMPLARY EMBODIMENT

With reference to the accompanying drawings, different exemplary embodiments of the construction according to the invention of a containment means with an aseptic working chamber are described in detail hereinbelow.

The following stipulation applies to the entirety of the further description. If a figure contains reference numerals for the purpose of clarity of the drawings and if it is clearly recognizable from the drawings but not mentioned in the immediately associated description text that the components are “repeating” components, reference is made, in the interests of brevity, to the explanation of such components in preceding figure descriptions.

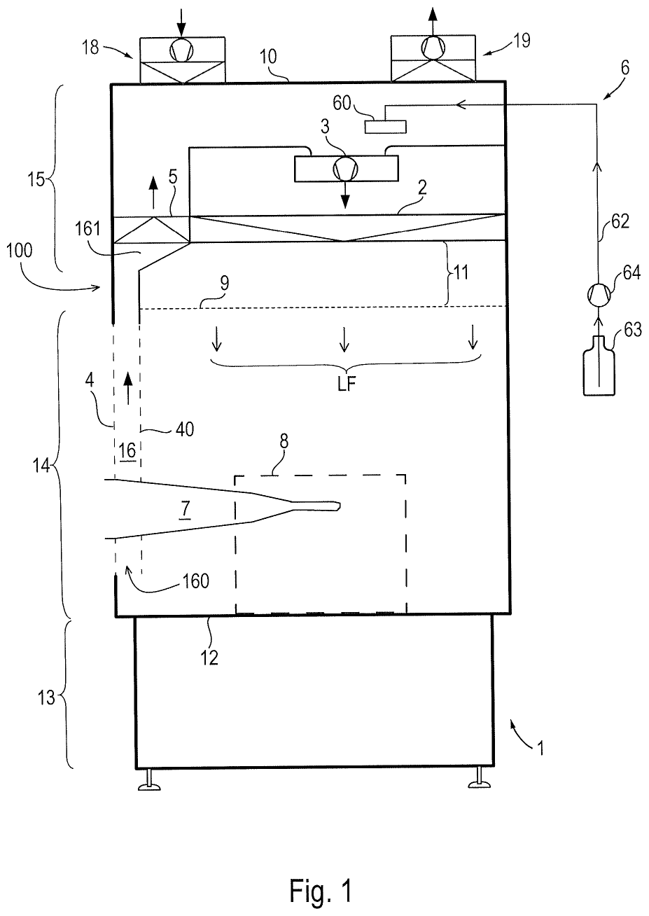

FIG. 1—First Embodiment

When seen vertically and from the bottom upwards, the containment means 1 is structured as the substructure 13, the working chamber 14 located above the substructure, the intermediate space 11 located above the working chamber, and the circulating air zone 15 arranged at the very top. The working chamber 13, the intermediate space 11 and the circulating air zone 15 are delimited towards the outside by the housing 10. The working chamber 14 is terminated at the bottom by the base 12 and at the top by the air guide element 9 in the form of a laminarizer—here a so-called CG distributor in the form of a mesh-like sterile air distributor. Connected to the circulating air zone 15 are an air supply unit 18 and the air outlet unit 19, each consisting of a fan and a filter. Via the air supply unit 18, the portion of conditioned fresh air is fed into the circulating air zone 15 to the air circulating in the containment means 1 in the operating mode and is discharged from the circulating air zone 15 via the air outlet unit 19. The circulating air fan 3 and the circulating air filter 2—here in the form of a plate filter—are arranged within the circulating air zone 15. In the operating mode, the circulating air fan 3 draws air from the circulating air zone 15 and conveys it through the circulating air filter 2 into the intermediate space 11 and then through the air guide element 9 stretched out beneath the intermediate space, in the form of a parallel displacement flow flowing downwards in the working chamber 14 in the direction towards the base 12, called a laminar flow LF for the sake of simplicity. In most cases, a processing machine 8, for example a filling machine, is installed in the working chamber 14, generally standing on the base 12.

The containment means 1 has at least a first returning air channel 16 which is surrounded on the front side 100 on the one hand by part of the housing 10 and the transparent front window 4 and on the other hand by the transparent channel window 40; the front window 4 and the channel window 40 are often together referred to in the industry as a double window. A pair of work gloves 7 is usually mounted on the front side 100, which allows an operator protected access to the working chamber 14. The returning air channel 16 extends from the vicinity of the base 12, where it has the inflow opening 160, into the circulating air zone 15. A returning air filter 5, currently in the form of a HEPA-plate-filter (high efficiency particulate air filter), is installed at the opening of the returning air channel 16 into the circulating air zone 15, specifically in this example in a projection 161 projecting into the circulating air zone 15. Owing to the conveying effect of the circulating air fan 3, the air circulates in the containment means 1 in the operating mode from the circulating air zone 15 via the working chamber 14 and through the returning air channel 16 back into the circulating air zone 15—with the partial replacement of air discharged via the air outlet unit 19 with conditioned fresh air introduced by means of the air supply unit 18. The returning air filter 5 thereby has a dual function. Firstly, any impure particles carried from the working chamber 14 with the returning air flow are separated out in the returning air filter 5. Secondly, no impure particles can pass from the circulating air zone 15—especially contrary to the circulating air flow—through the returning air filter 5, which also acts as a backflow preventer, via the returning air channel 16 into the working chamber 14.

For performing a decontamination phase, the containment means 1 is equipped with a decontamination arrangement 6. In the illustrated embodiment, a vaporizer 60 positioned in the circulating air zone 15 is provided, and decontamination agent, preferably an H₂O₂ solution, is supplied to the vaporizer from a storage container 63 via a material line 62 by means of a pump 64. In decontamination mode, the circulating air fan 3 is controlled at reduced power, e.g. 50%.

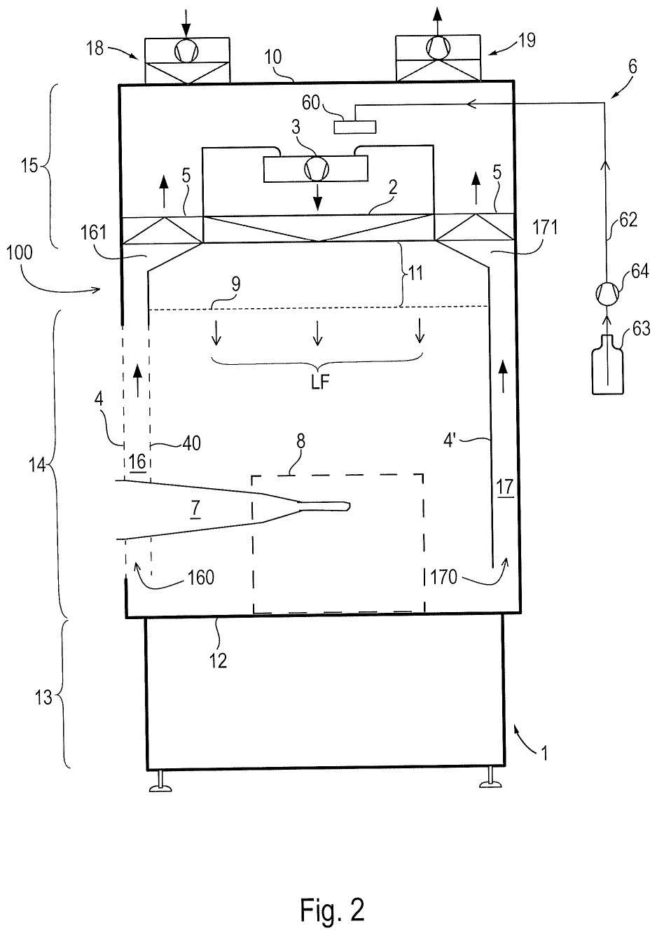

### FIG. 2—Second Embodiment

The only difference compared with the first embodiment according to FIG. 1 is that there is now additionally provided a second returning air channel 17 which comprises a wall portion of the housing 10 and a channel wall 4′ spaced apart therefrom and which has the inflow opening 170, which is again situated above and close to the base 12. A returning air filter 5 is also installed at the opening of the second returning air channel 17 into the circulating air zone 15, specifically in the projection 171 projecting into the circulating air zone 15. Such a second returning air channel 17 will be conceived for containment means 1 in which larger air volumes are to be processed.

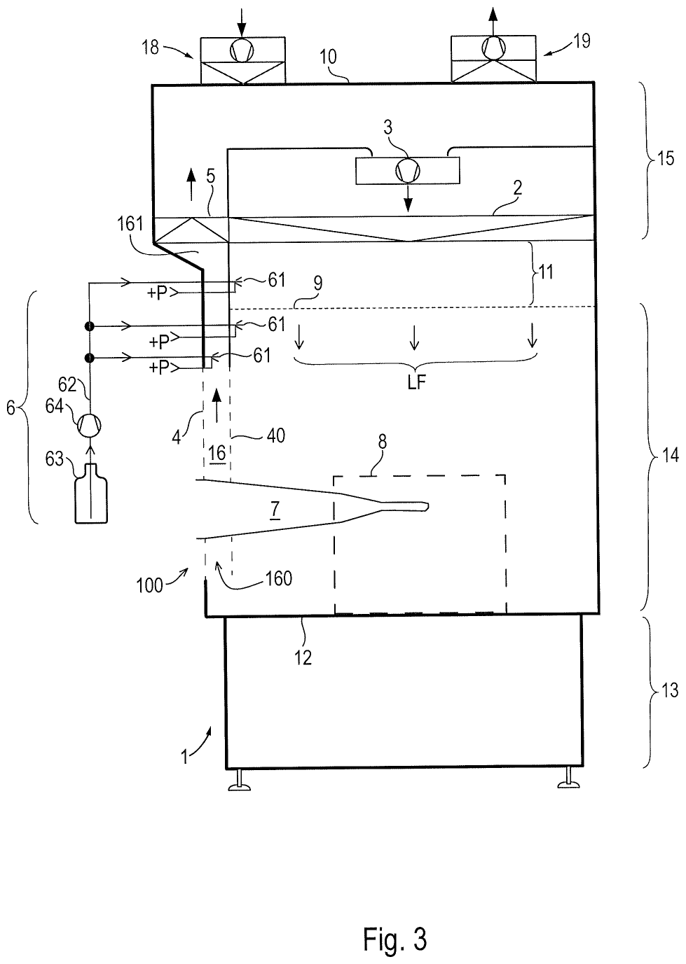

### FIG. 3—Third Embodiment

A difference compared with the first embodiment according to FIG. 1 concerns the form of the opening of the returning air channel 16 into the circulating air zone 15, specifically as a projection 161 which widens the circulating air zone 15 and in which the returning air filter 5 is installed, whereby the circulating air filter 2 can now extend in a fluidically advantageous manner over the entire width of the working chamber 14.

Furthermore, the decontamination arrangement 6 is now based on atomizing nozzles 61 by means of which the decontamination agent supplied via a material line 62 and a pump 64 and the supply of compressed air +P is sprayed in the form of an aerosol. Depending on the design of the containment means 1, at least one atomizing nozzle 61 directed into the working chamber 14 can be the most important. In the case of larger working chambers 14, it may be necessary to provide a plurality of atomizing nozzles 61 opening into the working chamber 14, which are advantageously arranged offset with respect to one another such that the aerosol introduced as the decontamination agent circulates in the working chamber 14. During operation of the containment means 1 in decontamination mode with the atomizing nozzles 61, the circulating air fan 3 is either switched off completely or down-regulated to such an extent that no volume flow is conveyed. Depending on the size and use of the containment means 1, a further atomizing nozzle 61 with a supplying material line 62 and compressed air +P can additionally be positioned in the intermediate space 11 and/or in the first returning air channel 16.

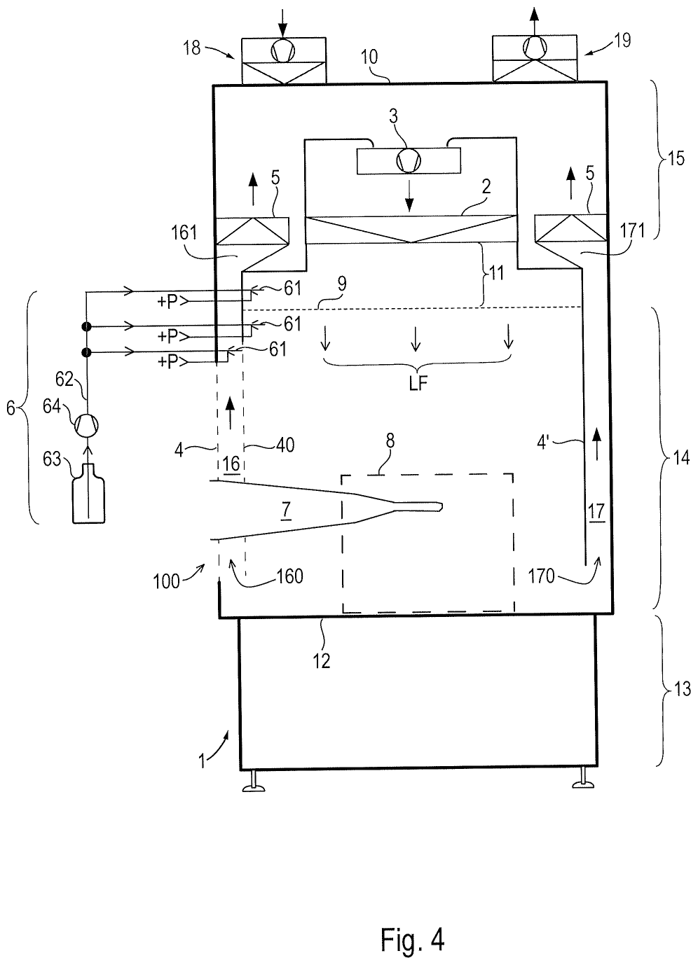

### FIG. 4—Fourth Embodiment

A mixed form of the construction of the containment means 1 is illustrated here.

The two returning air channels 16,17 with the respective inflow openings 160,170 and the two projections 161,171 projecting into the circulating air zone 15 each with a respective returning air filter 5 installed therein are taken from the second exemplary embodiment according to FIG. 2. The decontamination arrangement 6 with the atomizing nozzles 61, the material line 62 and the supply of compressed air +P is taken from the third embodiment according to FIG. 3.

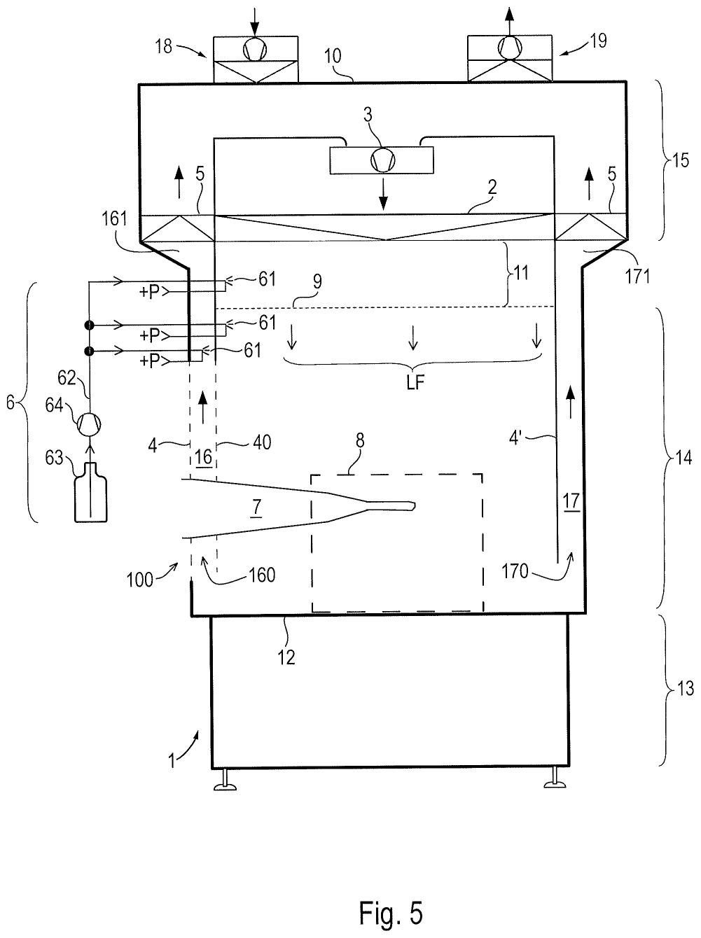

### FIG. 5—Fifth Embodiment

Compared to the preceding construction according to FIG. 4, the opening of each of the two returning air channels 16,17 into the circulating air zone 15 is now, as in principle according to the concept of FIG. 3, in the form of a projection 161,171 which widens the circulating air zone 15 and in each of which a returning air filter 5 is installed. The circulating air filter 2 can thus again be arranged in a fluidically optimal manner over the entire width of the working chamber 14.

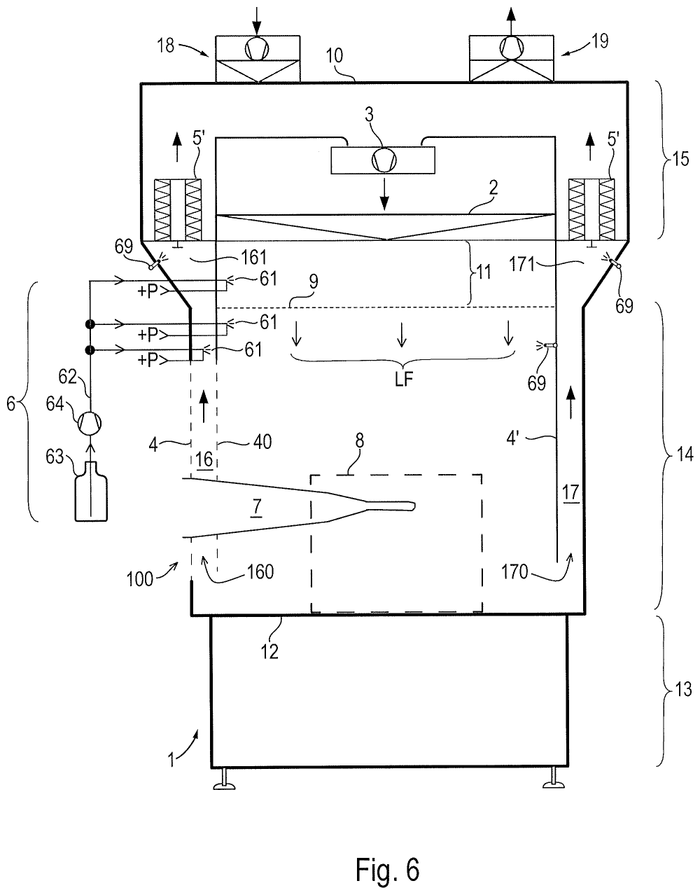

### FIG. 6—Sixth Embodiment

This construction is a modification of the construction according to FIG. 5; instead of the returning air filters 5 in the form of plate filters previously installed in each widening projection 161,171, exchangeable filter cartridges 5′ are now used.

Furthermore, a washing nozzle 69 opens into each of the projections 161,171, in each case beneath the filter cartridge 5′. In addition, at least one washing nozzle 69 is directed into the working chamber 14.

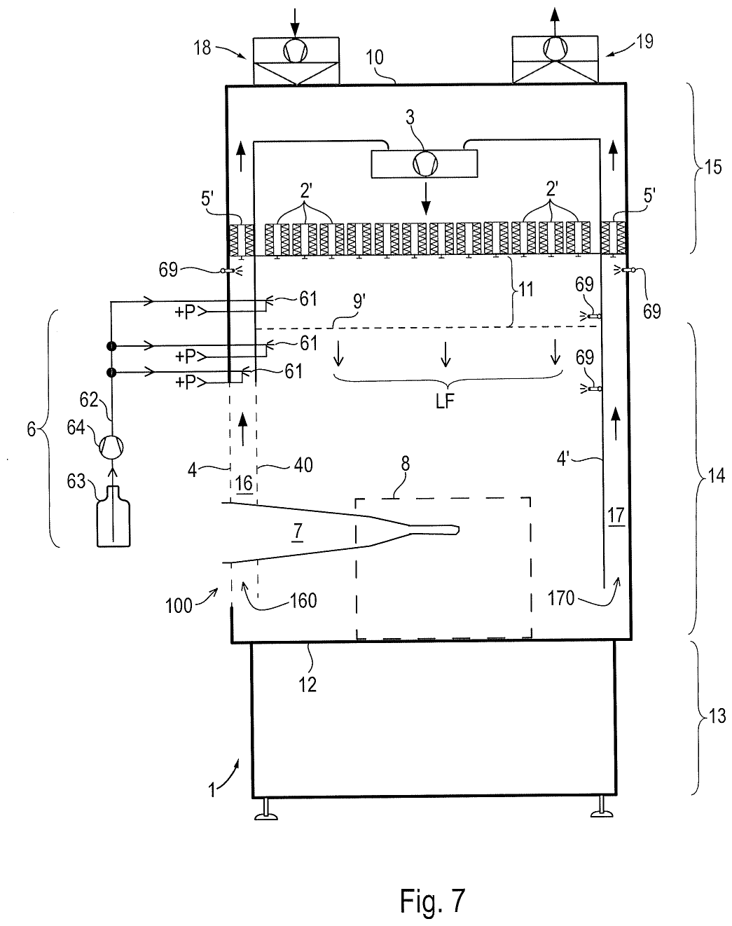

### FIG. 7—Seventh Embodiment

In this design, the two returning air channels 16,17 are again present, but they merge into the circulating air zone 15 without projections, virtually with a linear cross-section. An exchangeable filter cartridge as the returning air filter 5′ is arranged in each of the returning air channels 16,17, set back with respect to the transition into the circulating air zone 15. The circulating air filter 2′ now consists of exchangeable filter cartridges arranged in series. A washing nozzle 69 is directed into the returning air channel 16,17 in question, in each case again beneath the filter cartridge 5′, that is to say contrary to the direction of flow. In addition to the embodiment according to FIG. 6, at least one washing nozzle 69 directed into the intermediate space 11 is now also installed. In this case, owing to its greater durability with respect to the washing fluid, an air guide element 9′ in the form of a perforated plate is now used instead of the air guide element in the form of a CG-distributor used hitherto. The washing nozzles 69 directed into the returning air channels 16,17 and into the intermediate space 11 are fixedly installed, which is advantageous for handling, while the washing nozzle 69 directed into the working chamber 14 is preferably seated on a manual spray gun. The entire decontamination arrangement 6 is identical with the embodiments according to FIGS. 3 to 6.

1. A construction of a containment means with an aseptic working chamber and an associated decontamination arrangement, wherein:
a) the working chamber has a base at the bottom;
b) above the working chamber there is a circulating air zone, in which a circulating air filter with a circulating air fan is arranged;
c) the circulating air zone and the working chamber are delimited towards the outside by a housing;
d) at least a first returning air channel leads from the working chamber into the circulating air zone; and
e) a returning air filter is fluidically connected to the returning air channel, characterized in that
f) the returning air filter is arranged facing the circulating air zone.

2. The construction as claimed in claim 1, wherein the returning air filter is arranged at the opening of the returning air channel into the circulating air zone or in the returning air channel.

3. The construction as claimed in claim 2, wherein the opening of the returning air channel which holds the returning air filter:
a) is in the form of a projection which widens the circulating air zone or projects into the circulating air zone; or
b) merges into the circulating air zone without a projection, virtually with a linear cross-section.

4. The construction as claimed in claim 1, wherein:
a) the returning air filter is in the form of a plate filter or an exchangeable filter cartridge or a replaceable filter insert; and
b) the circulating air filter is in the form of a plate filter or an exchangeable filter cartridge or a replaceable filter insert.

5. The construction as claimed in claim 1, wherein:
a) a single returning air filter or a series of interacting returning air filters is provided; and
b) a single circulating air filter or a series of interacting circulating air filters is installed.

6. The construction as claimed in claim 1, wherein the at least a first returning air channel:
a) comprises as components a transparent front window and a transparent channel window spaced apart therefrom; and
b) has an inflow opening which is situated above and close to the base.

7. The construction as claimed in claim 1, wherein an air guide element extends between the circulating air zone and the working chamber and serves to generate a parallel displacement flow (LF) flowing downwards in the working chamber.

8. The construction as claimed in claim 1, wherein the decontamination arrangement has:
a) a vaporizer which is positioned in the circulating air zone and to which decontamination agent is supplied via a material line; or
b) at least one atomizing nozzle to which decontamination agent is supplied via a material line; wherein the at least one atomizing nozzle is directed into:
c) the working chamber; and/or
d) an intermediate space which is situated between the circulating air zone and the air guide element; and/or
e) the at least a first returning air channel.

9. The construction as claimed in claim 8, wherein during operation of the containment means in decontamination mode with the vaporizer in the decontamination arrangement, the circulating air fan is controlled with reduced power, e.g. 50%.

10. The construction as claimed in claim 8, wherein:
a) a series of atomizing nozzles directed into the working chamber is provided; and
b) during operation of the containment means in decontamination mode with the atomizing nozzles in the decontamination arrangement, the circulating air fan is down-regulated at least to such an extent that no volume flow is conveyed; wherein
c) the atomizing nozzles are arranged offset with respect to one another such that the aerosol introduced as the decontamination agent circulates in the working chamber.

11. The construction as claimed in claim 8, wherein:
a) the decontamination agent supplied via the material line to the vaporizer positioned in the circulating air zone is conveyed by means of a pump; or
b) the decontamination agent supplied via the material line to the at least one atomizing nozzle is conveyed by a pump and is sprayed in the form of an aerosol by means of compressed air (+P).

12. The construction as claimed in claim 1, wherein a washing nozzle is directed into:
a) the working chamber; and/or
b) an intermediate space which is situated between the circulating air zone and the air guide element; and/or
c) the at least a first returning air channel, specifically upstream of the returning air filter contrary to the direction of flow.

13. The construction as claimed in claim 12, wherein:
a) the washing nozzles directed into the returning air channel and/or into the intermediate space are fixedly installed; while
b) the washing nozzle directed into the working chamber is fixedly installed or is seated on a manual spray gun.

14. The construction as claimed in claim 1, wherein a second returning air channel is additionally provided, wherein:
a) the second returning air channel comprises a wall portion of the housing and a channel wall spaced apart therefrom and has an inflow opening which is situated above and close to the base;
b) the returning air filter is arranged facing the circulating air zone, specifically at the opening of the second returning air channel into the circulating air zone or in the second returning air channel;
c) the opening of the second returning air channel which holds the returning air filter can be in the form of a projection which widens the circulating air zone or projects into the circulating air zone, or it merges into the circulating air zone without a projection, virtually with a linear cross-section;
d) the returning air filter is in the form of a plate filter or an exchangeable filter cartridge or a replaceable filter insert;
e) the circulating air filter is in the form of a plate filter or an exchangeable filter cartridge or a replaceable filter insert;
f) a single returning air filter or a series of interacting returning air filters is provided; and
g) a single circulating air filter or a series of interacting circulating air filters is installed.

15. The construction as claimed in claim 14, wherein:
a) an atomizing nozzle is directed into the second returning air channel; and
b) a washing nozzle is directed into the second returning air channel, specifically is directed upstream of the returning air filter contrary to the direction of flow.
